(19) United States
(12) Patent Application Publication
(10) Pub. No.: US 2024/0221961 A1
(43) Pub. Date: Jul. 4, 2024

(54) Methods and Systems for Processing Pathology Data of a Patient For Pre-Screening Veterinary Pathology Samples

(71) Applicant: IDEXX Laboratories, Inc., Westbrook, ME (US)

(72) Inventors: Richard Luong, Westbrook, ME (US); Scott Bender, South Portland, ME (US); Eric Fish, Westrook, ME (US)

(21) Appl. No.: 18/391,169

(22) Filed: Dec. 20, 2023

Related U.S. Application Data
(60) Provisional application No. 63/436,063, filed on Dec. 29, 2022.

(51) Int. Cl. G16H 70/60; G16H 30/40

(52) U.S. Cl. CPC G16H 70/60; G16H 30/40

(57) ABSTRACT

An example computer-implemented method for processing pathology data includes providing a first graphical user interface for display on a first device, receiving pathology data associated with a patient, extracting a keyword from the pathology data, determining by executing a first machine-learning logic and based on the keyword extracted from the pathology data a pathology summary, providing a second graphical user interface for display on a second device presenting the pathology summary, and receiving a second input from the second graphical user interface. In response to receiving the second input at the second graphical user interface, the method includes providing for display at least one of: a background information module comprising data associated with the pathology summary; a contact information module comprising contact information of a pathologist associated with the pathology data; and an ordering module, which when initiated, generates an order for follow-on testing.

### CROSS REFERENCE TO RELATED APPLICATION

The present disclosure claims priority to U.S. Provisional Application No. 63/436,063, filed on Dec. 29, 2022, the entire contents of which are herein incorporated by reference.

### FIELD

The present disclosure relates generally to methods and systems for processing pathology data.

### BACKGROUND

Veterinary samples are typically collected at a point-of-care location, such as a veterinarian's office or the like. Interrogation of cellular samples may be beneficial to identify some conditions. The samples may be collected via blood draw, ear wax collection, biopsy, fine needle aspiration, or the like. The samples may then be positioned on a slide and stained for analysis (some slides may be created at a reference lab and some slides may be created at a point-of-care location). Analysis of samples requires specialized training, and many general practice veterinarians opt to have tissue samples analyzed by trained pathologists. Accordingly, following the manually staining process, the slide is digitized and the digitally formatted slide is reviewed by a pathologist to determine measurable pathology data useful for clinical decisions. In some instances, a pathologist may find it beneficial to have additional samples stained differently for observation of other features, or to have samples re-stained to improve color, intensity, or color balance. However, often additional samples are not available at a time of evaluation.

Pathology services typically generate a conventional pathology report, following analysis of samples, which are text-based reports requiring veterinarians to read, digest, and interpret pathology findings to make clinical decisions. In some instances, a pathology report is incomplete due to missing patient input data or unclear patient input data.

### SUMMARY

In an example, a computer-implemented method for processing pathology data is described. The method comprises providing a first graphical user interface for display on a first device, receiving a first input from the first graphical user interface and the first input comprising pathology data associated with a patient, extracting, by a processor, a keyword from the pathology data, and determining, by the processor executing a first machine-learning logic and based on the keyword extracted from the pathology data, a pathology summary, wherein the first machine-learning logic is trained using veterinary pathology training data labeled with corresponding diagnostic results. The method also comprises providing a second graphical user interface for display on a second device presenting the pathology summary, receiving a second input from the second graphical user interface, and in response to receiving the second input at the second graphical user interface, providing for display at least one of: a background information module comprising data associated with the pathology summary, a contact information module comprising contact information of a pathologist associated with the pathology data, and an ordering module, which when initiated, generates an order for follow-on testing.

In another example, a server is described comprising one or more processors, and non-transitory computer readable medium having stored therein instructions that when executed by the one or more processors, causes the server to perform functions. The functions comprise providing a first graphical user interface for display on a first device, receiving a first input from the first graphical user interface and the first input comprising pathology data associated with a patient, extracting a keyword from the pathology data, and determining, by executing a first machine-learning logic and based on the keyword extracted from the pathology data, a pathology summary, wherein the first machine-learning logic is trained using veterinary pathology training data labeled with corresponding diagnostic results. The functions also comprise providing a second graphical user interface for display on a second device presenting the pathology summary, and receiving a second input from the second graphical user interface, and in response to receiving the second input at the second graphical user interface, providing for display at least one of: a background information module comprising data associated with the pathology summary, a contact information module comprising contact information of a pathologist associated with the pathology data, and an ordering module, which when initiated, generates an order for follow-on testing.

In another example, a non-transitory computer readable medium is described having stored thereon instructions, that when executed by one or more processors of a computing device, cause the computing device to perform functions. The functions comprise providing a first graphical user interface for display on a first device, receiving a first input from the first graphical user interface and the first input comprising pathology data associated with a patient, extracting a keyword from the pathology data, and determining, by executing a first machine-learning logic and based on the keyword extracted from the pathology data, a pathology summary, wherein the first machine-learning logic is trained using veterinary pathology training data labeled with corresponding diagnostic results. The functions also comprise providing a second graphical user interface for display on a second device presenting the pathology summary, receiving a second input from the second graphical user interface, and in response to receiving the second input at the second graphical user interface, providing for display at least one of: a background information module comprising data associated with the pathology summary, a contact information module comprising contact information of a pathologist associated with the pathology data, and an ordering module, which when initiated, generates an order for follow-on testing.

The features, functions, and advantages that have been discussed can be achieved independently in various examples or may be combined in yet other examples. Further details of the examples can be seen with reference to the following description and drawings.

### BRIEF DESCRIPTION OF THE FIGURES

Examples and descriptions of the present disclosure will be readily understood by reference to the following detailed description of illustrative examples when read in conjunction with the accompanying drawings, wherein:

FIG. 1 illustrates an example of a system, according to an example implementation.

FIG. 2 illustrates an example computer system of the system of FIG. 1, according to an example implementation.

FIG. 3 illustrates an example of a structured input format of a GUI of the system of FIG. 1, according to an example implementation.

FIG. 4 illustrates an example of a structured output format of the GUI of the system of FIG. 1, according to an example implementation.

FIG. 5 illustrates another example of a structured output format of the GUI of the system of FIG. 1, according to an example implementation.

FIG. 6 illustrates another example of a structured output format of the GUI of the system of FIG. 1, according to an example implementation.

FIG. 7 shows a flowchart of a method for processing pathology data, according to an example implementation.

### DETAILED DESCRIPTION

Disclosed examples will now be described more fully hereinafter with reference to the accompanying drawings. Several different examples are described herein, but embodiments should not be construed as limited to the examples set forth herein. Rather, these examples are described so that this disclosure will be thorough and complete and will fully convey the scope of the disclosure to those skilled in the art.

Generation and synthesis of pathology data is generally a manual process performed by a pathologist, and can be a time-consuming process. Systems and methods described herein include a computer-implemented method for processing pathology data in which a first graphical user interface is display on a first device for input of pathology data associated with a patient according to a structured input format. With inputs received in a known format, the inputs are processed by executing a first machine-learning logic that is trained using veterinary pathology training data labeled with corresponding diagnostic results to generate a pathology summary. The pathology summary is sent to a second graphical user interface for display on a second device, and in response to receiving a second input at the second graphical user interface, a corresponding follow-on action occurs enabling the patient owner to have more access and control of the testing processes.

Systems and methods described also consider that some example pathology data may be incomplete or inconclusive. As such, some implementations of the disclosure provide for creation of a digitally-stained slide based on a prior collected sample of the patient to simulate a staining of the sample to generate additional pathology data for analysis.

Implementations of this disclosure provide technological improvements that are particular to computer technology, for example, those concerning operation and control of graphical user interfaces. Computer-specific technological problems, such as receiving pathology data, can be wholly or partially solved by implementations of this disclosure. For example, implementation of this disclosure allows for pathology data to be input into a structural input format, and in some instances, portions of the pathology data are digitally generated (e.g., a digitally-stained slide) for creation of further data for input.

The systems and methods of the present disclosure further address problems particular to computer devices and operation of diagnostic instruments, for example, those concerning analysis of pathology data. Utilizing machine-learning algorithms, trained on manually labeled pathology data, enables a more immediate and normalized analysis of the data. Thus, analysis of the pathology data can occur in a manner that is efficient and takes into account patients' unique needs. Moreover, in embodiments according to the present disclosure, pathology summaries are provided in a manner allowing follow-on testing to be immediately determined and scheduled. Implementations of this disclosure can thus introduce new and efficient improvements in the ways in follow-on testing is scheduled by the central computing device for use of the diagnostic instruments in an efficient manner.

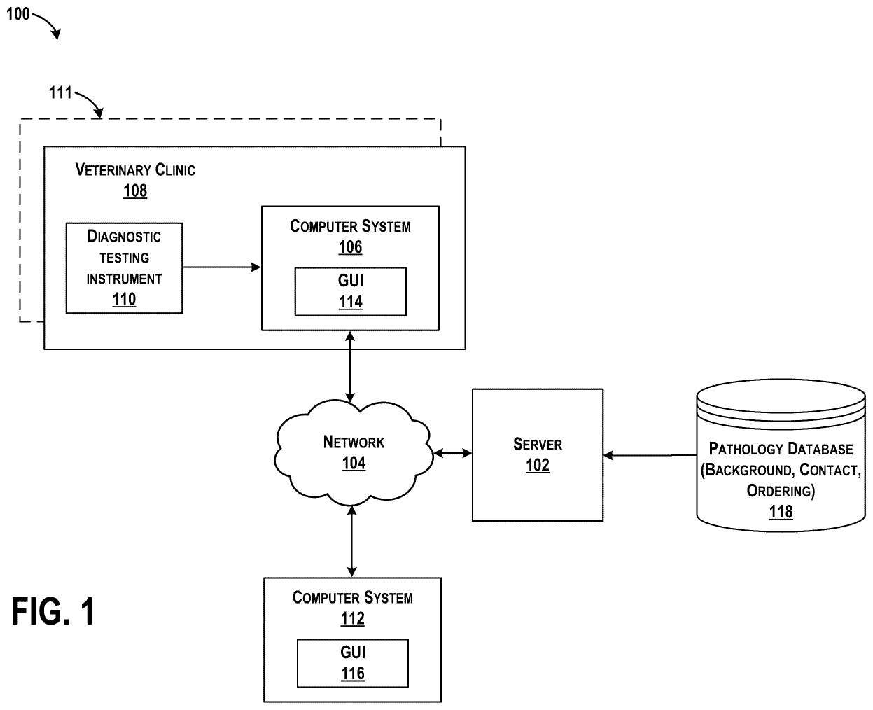

Referring now to the figures, FIG. 1 illustrates an example of a system 100, according to an example implementation. The system 100 includes a server 102 accessible through a network 104 by multiple different computer systems. In embodiments, one of the computer systems includes a computer system 106 residing at a veterinary clinic 108. In the embodiment depicted in FIG. 1, the veterinary clinic 108 includes a diagnostic testing instrument 110 to perform diagnostic testing of veterinary patients, the diagnostic testing instrument 110 communicatively coupled to the computer system 106. In embodiments, the diagnostic testing instrument 110 outputs test results to the computer system 106. While one veterinary clinic 108 is depicted in FIG. 1, it should be understood that this is merely an example, and systems 100 according to the present disclosure can include any suitable number of veterinary clinics and associated computer systems, such as a second veterinary clinic 111 that includes the same components as the veterinary clinic 108. As referred to herein, the term “veterinary clinics” includes any entity at which non-human animals receive medical care, and can include brick and mortar locations, mobile clinics, on-line virtual clinics, pop-up clinics, and the like.

In addition, while the example depicted in FIG. 1 include one diagnostic testing instrument 110, it should be understood that this is merely an example, and embodiments according to the present disclosure can include any suitable number of diagnostic testing instruments associated with the veterinary clinic 108. Examples of the diagnostic testing instrument 110 include any one or combination of veterinary analyzers operable to conduct a diagnostic test of a sample of a patient (e.g., operable to determine hemoglobin amounts in a blood sample, operable to analyze a urine sample, and/or the like). Such veterinary analyzers include, for example and without limitation, a clinical chemistry analyzer, a hematology analyzer, a urine analyzer, an immunoassay reader, a sediment analyzer, a blood analyzer, a microscopic analyzer, a digital radiology machine, and/or the like. In one example, the computing system 106 is in communication with a veterinary analyzer of the diagnostic testing instrument 110 and is operable to receive diagnostic information from veterinary analyzer. The diagnostic testing instrument 110 outputs signals, such as signals indicative of diagnostic test results or other information, to the computing system 106.

The server 102 receives an input from the computer system 106 over the network 104. The input, in embodiments, includes medical record information associated with a veterinary patient, a veterinary patient identifier unique to the veterinary patient, data associated with test results output from the diagnostic testing instrument 110, and/or the like.

The system 100, in embodiments, includes a second computer system 112 connected to the network 104 for access to the server 102. The second computer system 112 is distinct and different from the computer system 106. In some examples, the computer system 112 resides at a location different from the computer system 106. In other examples, the computer system 112 resides at the veterinary clinic 108 as well, such as when the computer system 112 is a customer computer system.

In the system 100, the network 104 (e.g., Internet) provides access to the server 102 for all network-connected components. In some examples, more components of the system 100 may be in communication with the network 104 to access the server 102. Communication with the server 102 and/or with the network 104 may be wired or wireless communication (e.g., some components may be in wired Ethernet communication and others may use Wi-Fi communication). In some examples, the network 104 provides access for the computer system 112 to communicate with the computer system 106 directly as well.

The system 100 enables a method for processing pathology data. The computer system 106 includes a graphical user interface (GUI) 114 for display, and is operable to receive inputs. The inputs, in embodiments, can take the form of pathology data associated with a patient. Within examples, the pathology data includes any of outputs from the diagnostic testing instrument 110, information about the patient (e.g., gender, breed, age, etc.), and/or other test results as well of medical tests performed on the patient. The computer system 106, via access to the server 102, generates a structured template for display on the GUI 114 based on inputs provided at the GUI 114. The structured template comprises fields displayed for input in a rules-based manner such that the GUI 114 displays a field for further input and each subsequent field provided for display is based on the further input provided in a prior field.

In embodiments, the computer system 112 includes a GUI 116. The computer system 112 receives data, from the computer system 106 or the server 102. The GUI 116, in some embodiments, receives user input.

Information input to the GUI 116 or 114 is processed by the computer system 106 and/or provided to the server 102 for processing. Example processing includes extracting a keyword from the pathology data, and then determining a pathology summary. The pathology summary includes an analysis of the pathology data, based on the extracted keyword, to provide interpretations and perhaps diagnosis related to the pathology data for the patient. In one example, the pathology summary is determined by executing a machine-learning logic, based on the keyword extracted from the pathology data, where the machine-learning logic is trained using veterinary pathology training data labeled with corresponding diagnostic results (described more fully below).

The GUI 114 is operable to receive data from the computer system 112, and in response to receives an data at the computer system 112, the GUI 116 provides an updated display including at least one of a background information module comprising data associated with the pathology summary, a contact information module comprising contact information of a pathologist associated with the pathology data, and an ordering module, which when initiated, generates an order for follow-on testing. Based on the input received, the GUI 114 updates the display accordingly. The computer system 106 receives information for updating the GUI 114 from the server 102, which has access to a pathology database 118 that stores information for background information of test results, contact information of pathologists, and order information for follow-on testing. The GUI 114 thus provides functionality to the customer at the computer system 106, for example, to order follow-on testing directly from pathology summary.

The system 100 provides advantages to the customer, which may be a veterinarian at the veterinary clinic 108, a veterinarian technician at the veterinary clinic 108, and/or an owner of the veterinary patient, including explanation of a pathology reports as well as digital access to information associated with the report. The pathology summary provided to the computer system 106 for the customer is a contextual and interactive report, for example.

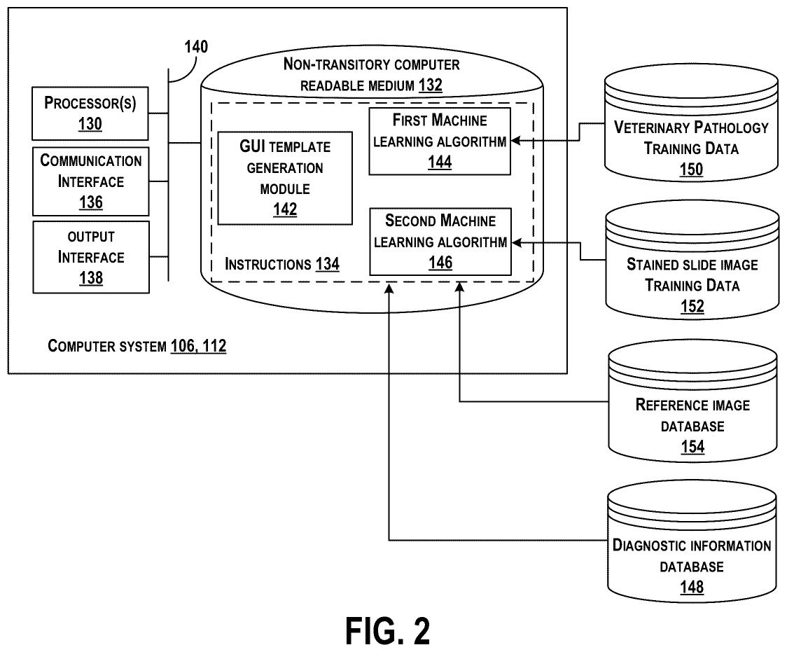

FIG. 2 illustrates an example of the computer system 112 in FIG. 1, according to an example implementation. Within examples herein, functions described for processing pathology data are performed by the computer system 112, by the server 102, or by a combination of the computer system 112 and the server 102. Thus, although FIG. 2 illustrates the computer system 112, the components of the server 102 and/or the computer system 106 are the same as the components of the computer system 112 and the illustration in FIG. 2 additionally represents similar components of the server 102 and/or the computer system 106, for example, depending on where a function is programmed to be performed in a specific example.

The computer system 112 includes one or more processor(s) 130, and non-transitory computer readable medium 132 having stored therein instructions 134 that when executed by the one or more processor(s) 130, causes the computer system 112 to perform functions for operation, management, and control of diagnostic instruments, for generation of a GUI, and for processing pathology data, for example.

In some embodiments, the computer system 112 includes a communication interface 136, an output interface 138, and one or more components of the computer system 112 is connected to a communication bus 140. In some embodiments, the computer system 112 includes hardware to enable communication within the computer system 112 and between the computer system 106 and other devices (not shown). The hardware may include transmitters, receivers, and antennas, for example. The computer system 112 may further include a display (not shown).

The communication interface 136 may be a wireless interface and/or one or more wireline interfaces that allow for both short-range communication and long-range communication to one or more networks or to one or more remote devices. Such wireless interfaces may provide for communication under one or more wireless communication protocols, Bluetooth, WiFi (e.g., an institute of electrical and electronic engineers (IEEE) 802.11 protocol), Long-Term Evolution (LTE), cellular communications, near-field communication (NFC), and/or other wireless communication protocols. Such wireline interfaces may include an Ethernet interface, a Universal Serial Bus (USB) interface, or similar interface to communicate via a wire, a twisted pair of wires, a coaxial cable, an optical link, a fiber-optic link, or other physical connection to a wireline network. Thus, the communication interface 136 may be configured to receive input data from one or more devices, and may also be configured to send output data to other devices.

The non-transitory computer readable medium 132 may include or take the form of memory, such as one or more computer-readable storage media that can be read or accessed by the one or more processor(s) 130. The non-transitory computer readable medium 132 can include volatile and/or non-volatile storage components, such as optical, magnetic, organic or other memory or disc storage, which can be integrated in whole or in part with the one or more processor(s) 130. In some examples, the non-transitory computer readable medium 132 can be implemented using a single physical device (e.g., one optical, magnetic, organic or other memory or disc storage unit), while in other examples, the non-transitory computer readable medium 132 can be implemented using two or more physical devices. The non-transitory computer readable medium 132 thus is a computer readable storage, and the instructions 134 are stored thereon. The instructions 134 include computer executable code.

The one or more processor(s) 130 may be general-purpose processors or special purpose processors (e.g., digital signal processors, application specific integrated circuits, etc.). The one or more processor(s) 130 may receive inputs from the communication interface 136 (e.g., x-ray images), and process the inputs to generate outputs that are stored in the non-transitory computer readable medium 132. The one or more processor(s) 130 can be configured to execute the instructions 134 (e.g., computer-readable program instructions) that are stored in the non-transitory computer readable medium 132 and are executable to provide the functionality of the computer system 106 described herein.

In embodiments, the output interface 138 outputs information for transmission, reporting, or storage, and thus, the output interface 138 may be similar to the communication interface 136 and can be a wireless interface (e.g., transmitter) or a wired interface.

Within examples, the instructions 134 may include specific software for performing the functions including a GUI template generation module 142, a first machine-learning algorithm 144 (e.g., for determining a pathology summary), and a second machine-learning algorithm 146 (e.g., for creating virtual or digitally-stained slides).

The GUI template generation module 142 is executed to generate a GUI for display that includes a structured input format and/or a structured output format. The structured input format of the GUI includes fields to be populated by inputs, where a subsequently displayed field is selected based on an input provided in a prior field. Thus, data entered into the GUI is decision point for selection of a next field to be displayed. In one example, a decision tree is used to generate the structured input format.

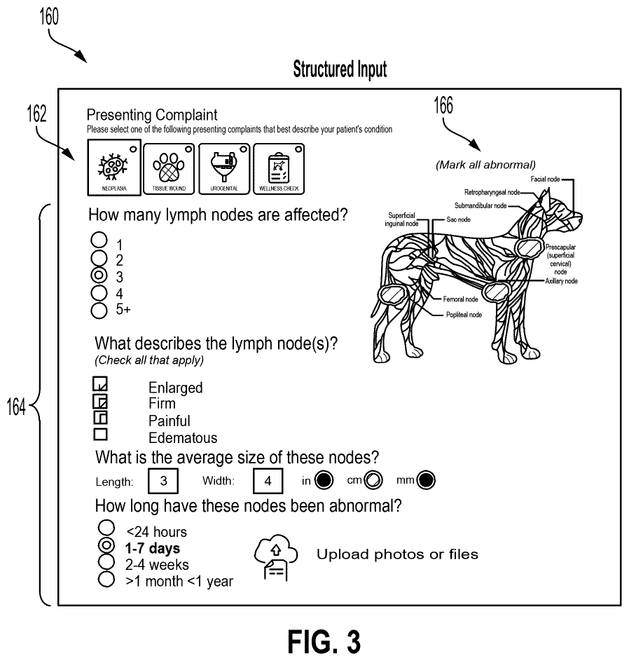

FIG. 3 illustrates an example of a structured input format 160 of the GUI 114 in FIG. 1, according to an example implementation. The structured input format 160 begins by displaying a number of categories of complaints 162 for selection, such as to request the veterinarian to input a complaint corresponding to a condition of the patient. In the example in FIG. 3, a complaint for neoplasia is selected.

Next, based on selection of neoplasia, the GUI template generation module 142 generates a set of questions 164 corresponding to neoplasia. Following receiving additional inputs to the set of questions 164, the GUI template generation module 142 generates an image 166 for annotation/selection of areas on the patient presenting possible signs of abnormalities. Depending on an input provided and a condition being analyzed, the GUI template generation module 142 continues to generate follow-on input fields accordingly.

The structured input format 160 in FIG. 3 illustrates all fields simultaneously for illustration purposes. However, in some examples, each subsequent field is displayed upon receiving an input in a prior field, since each subsequent field is determined based on the prior received input. For instance, in FIG. 3, upon receiving selection of a category of complaints, then the set of questions 164 is determined and displayed. Thus, initially, the structured input format 160 only includes the initial field to present the number of categories of complaints 162 for selection.

Referring back to FIG. 2, computer system 106 has access to or is in communication with a diagnostic information database 148 that stores information of all types of conditions and questions to present in the GUI 114 based on an identified condition. Thus, the GUI template generation module 142 receives information from the diagnostic information database 148 for use in generating information and questions for display on the GUI 114. Once an input is provided to the GUI 114, the GUI template generation module 142 accesses the diagnostic information database 148 to filter possible questions that are relevant to the provided input, and then populates the structured input format 160 accordingly. As a result, in one example, the templates and follow-on questions are pre-stored in the diagnostic information database 148. The GUI template generation module 142 offers advantages, in contrast to a free form text input system, in which inputs are normalized and focused so that all necessary information for generation of a possible diagnosis is received.

In embodiments, information received via GUI 114 and/or 116 is synthesized to form a pathology report in a structured output format.

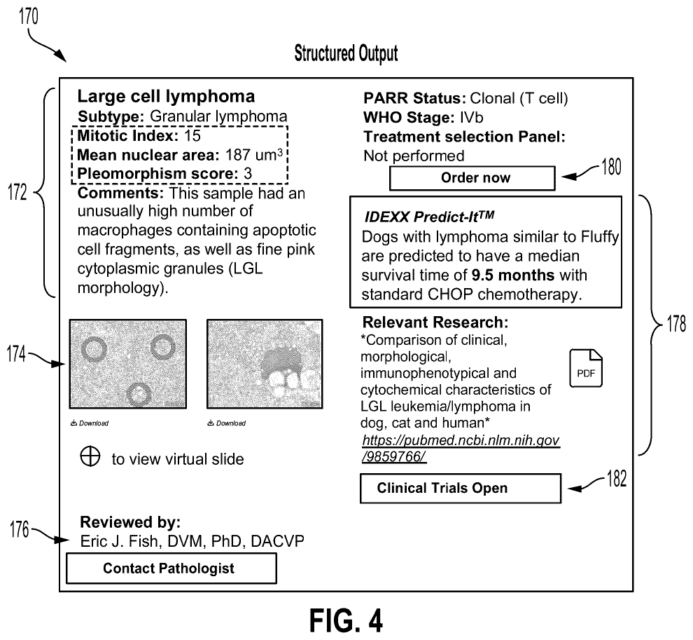

FIG. 4 illustrates an example of a structured output format 170 of the GUI 114 in FIG. 1, according to an example implementation. The structured output format 170 is generated by the GUI template generation module 142 for display on the GUI 116 and include a diagnosis field 172 including information about the diagnosis of the patient based on the data input, an image field 174 showing images of stained-slides of samples from the patient, a contact information module 176 including contact information of a pathologist associated with the pathology data, a background information module 178 including data associated with the pathology summary, an ordering module 180 selectable to generate an order for follow-on testing, and a clinical trials module 182 selectable to schedule a clinical trial.

Thus, the structured output format 170 provided in the GUI 114 enables an input to be received at the GUI 116 within one of the modules.

Referring back to FIG. 2, the first machine-learning algorithm 144 is executable to analyze the keyword extracted from the pathology data for generation of a pathology summary. The first machine-learning algorithm 144 is trained using veterinary pathology training data labeled with corresponding diagnostic results. Using the example structured input 160 shown in FIG. 3, the pathology data associated with the patient includes selection of a complaint, number of lymph nodes affected, how the lymph nodes are affected, size of the lymph nodes, length of time of abnormality of the lymph nodes, and any photos or files uploaded. The computer system 106 extracts, by the processor 130, a keyword from the pathology data. In this example, the input pathology data is processed by the first machine-learning algorithm 144 to determine matched diagnostic outputs to all of the information input, and the keyword may be any word from information input that matches to an output, such as lymphoma. The computer system 106 determines the pathology summary by the executing the first machine-learning logic 144 and based on the keyword extracted from the pathology data. The pathology summary is generated again by matching to the veterinary pathology training data 150 labeled with corresponding diagnostic results.

In one example, a keyword includes a species (i.e., cat or feline), and a species-specific keyword drives a distinct set of structured or predictive outcome options (i.e., cat drive a distinct set of possible lymphoma type outcomes compared to dog). In another example, a keyword includes a breed (i.e., Bernese Mountain Dog) and a breed-specific keyword drives a distinct set of structured or predictive outcome options (i.e., systemic histiocytosis).

In one example, the pathology data associated with the patient and input into the computer system 106 or 112 includes a digital copy of a stained slide. For example, a sample can be obtained from the patient, and the sample is manually-stained and then digitized for input. The stained slide contains measurable pathology data. The computer system 106 determines the pathology summary by the executing the first machine-learning logic 144 and based on the stained slide again by matching to the veterinary pathology training data 150 labeled with corresponding diagnostic results.

When images are provided as input, such as from a stained slide, the computer system 112 determines the pathology summary by the executing the first machine-learning logic 144 to generate a mitotic count. The computer system 112 also includes or has access to a reference image database 154 that stores reference images with corresponding labels of a mitotic count. The reference images include selected region that have been manually labeled for indicated of mitotic cells. Thus, the computer system 112 receives a pathology image of the patient (such as a stained slide image), and compares the pathology image of the patient with reference images in the reference image database 154 to determine the mitotic count. The mitotic count may be considered an approximate cell count based on a comparison of images (some of which are labeled), for example.

In one example, to manually determine the mitotic count, a trained pathologist reviews the image and recognized a dividing cell by a presence of a range of nuclear changes (i.e., a mitotic figure is nucleus undergoing division). The pathologist then counts the number of mitotic figures in a predetermined area of tissue in question, and the result is the mitotic count. The images are labeled and tagged with associated mitotic counts to create the reference images used by the computer system 112 to determine the mitotic count using the machine learning algorithm.

The mitotic count, when present, is then provided in the pathology summary, as shown in FIG. 4 in the diagnosis field 172, in one example. In some other examples, a mitotic index or ratio between number of cells in a population undergoing mitosis to a total number of cells in a population is provided (e.g., in FIG. 4, mitotic index 15; representative of how many cells in the tissue are mitotically active giving an indication of a potential for malignancy). Other measurable metrics are provided when present, such as mean nuclear area and pleomorphism score.

Thus, in embodiments, the first machine-learning logic 144 processes an input image to determine whether any cell include a mitotic figure, by image processing and comparison to reference images in the reference image database 154, an estimation or approximation of similar cells in the input image is determined for an interpretation-aid, for example. Execution of the first machine-learning algorithm 144 to perform analysis of inputs removes any human pathologist bias and generates normalized results for all inputs.

The second machine-learning algorithm 146 of the computer system 106 or 112 is executable by the processor to create a digitally-stained slide, and the second machine-learning logic 146 is trained using images of a plurality of physically stained slides labeled with attributes for a stain used on a sample. A digitally-stained slide is used herein to represent a slide digitally created by simulating manual staining of a sample. A digitally-stained slide may be considered a virtually created stained slide generated by execution of the second machine-learning algorithm 146 based on optically-based physicochemical techniques. Virtual versions of routine stains (e.g., hematoxylin-and-eosin (“H&E”)) can be auto-generated for all cases, while other specialized stains can be generated on-demand. Many technological advantages of virtual staining are possible including improved staining quality of digitized slides (staining color, intensity, and color balance can be consistently applied to all slides, regardless of clinic origin or processing lab); remove a need for manually-based staining equipment, staining consumables, and labor; provide an opportunity for on-demand virtual staining (reducing wait times for pathologist and client veterinarian/pet owner); and improved interpretative confidence (the base slide can be used to generate all virtual-stains, meaning that the same based slide is being assessed with all stains).

Diagnostic pathology slides can contain observations that are measured by the pathologist, such as diagnostic quality, mitotic count, histologic-free margins, or presence of organisms. The second machine-learning algorithm 146 is executable, based on training using the stained slide image training data 152, to generate digitally-stained slides for measurable data for pathologist review, validation and/or approval. Certain measurable data could be used by the second machine-learning algorithm 146 to pro-actively offer interpretation prediction (with confidence measures) and to populate the pathology summary.

Thus, within examples, the second machine-learning algorithm 146 creates a digitally-stained slide based on a prior physically collected sample of the patient and reference to the stained slide image training data 152, and the computer system 106 provides within the pathology summary for display on the GUI 116 an analysis of the digitally-stained slide. In this example, the digitally-stained slide is considered an input into the structured input format 160 for processing.

In one example, to create the digitally-stained slide, a prior collected image of a sample of the patient, which is a label-free tissue section, is transferred into an image that is histologically stained. The second machine-learning algorithm 146 is trained using the stained slide image training data 152 that includes pairs of image data (label-free tissue images and corresponding brightfield images acquired after staining). Features and characteristics in the label-free tissue images are modified in a same as features and characteristics in the training data to create the digitally-stained slide illustrating a simulated effect of applying the stain to the tissue. Application of the stained slide image training data 152 maps usage of all stains on different features of tissue, and such mapping is then applied to the prior collected image of the sample of the patient. Thus, one physical slide of a sample of a patient is used to digitally create other stained slides.

When creating the digitally-stained slide, the second machine-learning algorithm 146 receives input as to which stain to use. In some instances, the digitally-stained slide is created based on a mix of stains. A default stain is applied first to all slides, and then depending on inputs received (e.g., organ or tissue type; a keyword in the complaint; a type of testing order from the veterinarian; end-user pathologist input), other staining is determined for use.

In other instances, a look-up table is referenced to determine an appropriate stain to use for an appropriate slide creation.

In some examples, based on an analysis of the input at the GUI 114 or 116, the computer system 106 determines that a stained slide analysis of a sample from the patient is needed. For instance, a default stain is set to always be required (e.g., hematoxylin and eosin for biopsy slides; Wright-Giemsa stain for cytology slides). In either of these examples, if the complaint includes the keyword “bacteria” or the pathologist sees something suspicious to be “bacteria”, then a virtual Gram stain is determined for use to help positively identify and sometimes help determine any bacteria that are present.

Subsequently, the second machine-learning algorithm 146 is executed to create a digitally-stained slide based on a prior collected sample of the patient for input to the structured input format 160, and an analysis of the digitally-stained slide is provided within the pathology summary for display on the GUI 116, for example.

In one example, the GUI template generation module 142 analyzes inputs and determines that a possible condition is present in the patient. But, to conclusively provide a diagnosis, an analysis of a sample is desirable, and thus, the second machine-learning algorithm 146 is executed to create the digitally-stained slide for analysis. Analysis of the digitally-stained slide includes capturing measurable data useful for mapping to a certain diagnosis. In this example, the analysis of the digitally-stained slide is a pre-screen for a condition, since the analysis is based on a simulation of measurable data on what a physically stained slide would show. Thus, in some instances, when the pre-screen results in a certain possible diagnosis, the pathology summary includes a recommendation for follow-on testing to retrieve a physical sample of the patient and confirm results of the digitally-stained slide analysis by having a pathologist perform a manual stain of the physical sample.

In yet another example, based on an analysis of the input at the GUI 114 where the input includes an image of a manually stained slide including a physical sample of the patient, the computer system 106 determines that a digitally-stained slide analysis is needed. In this example, the input stained slide image includes some defect, such as being blurry, not including enough cells, etc., and thus, the digitally-stained slide is generated to create an improved image for analysis so that a mitotic count can be accurately performed.

Referring to the first machine-learning algorithm 144 and the second machine-learning algorithm 146, many types of functionality and neural networks can be employed to perform functions of the machine-learning algorithms. In one example, the first machine-learning algorithm 144 and the second machine-learning algorithm 146 use statistical models to generate the outputs without using explicit instructions, but instead, by relying on patterns and inferences by processing associated training data.

The first machine-learning algorithm 144 and the second machine-learning algorithm 146 can thus operate according to machine-learning tasks as classified into several categories. In supervised learning, the first machine-learning algorithm 144 and the second machine-learning algorithm 146 build a mathematical model from a set of data that contains both the inputs and the desired outputs. The set of data is sample data known as the “training data”, in order to make predictions or decisions without being explicitly programmed to perform the task. For example, the first machine-learning algorithm 144 utilizes the veterinary pathology training data 150 and the second machine-learning algorithm 146 uses stained slide image training data 152.

In another category referred to as semi-supervised learning, the first machine-learning algorithm 144 and the second machine-learning algorithm 146 develop mathematical models from incomplete training data, where a portion of the sample input does not have labels. A classification algorithm can then be used when the outputs are restricted to a limited set of values.

In another category referred to as unsupervised learning, the first machine-learning algorithm 144 and the second machine-learning algorithm 146 builds a mathematical model from a set of data that contains only inputs and no desired output labels. Unsupervised learning algorithms are used to find structure in related training data, such as grouping or clustering of data points. Unsupervised learning can discover patterns in data, and can group the inputs into categories.

Alternative machine-learning algorithms may be used to learn and classify types of follow testing to consider for generating the recommendations, such as deep learning though neural networks or generative models. Deep machine-learning may use neural networks to analyze prior test results through a collection of interconnected processing nodes. The connections between the nodes may be dynamically weighted. Neural networks learn relationships through repeated exposure to data and adjustment of internal weights. Neural networks may capture nonlinearity and interactions among independent variables without pre specification. Whereas traditional regression analysis requires that nonlinearities and interactions be detected and specified manually, neural networks perform the tasks automatically.

Still other machine-learning algorithms or functions can be implemented to generate the recommendations, such as any number of classifiers that receives input parameters and outputs a classification (e.g., attributes of the image). Support vector machine, Bayesian network, a probabilistic boosting tree, neural network, sparse auto-encoding classifier, or other known or later developed machine-learning algorithms may be used. Any semi-supervised, supervised, or unsupervised learning may be used. Hierarchal, cascade, or other approaches may be also used.

The first machine-learning algorithm 144 and the second machine-learning algorithm 146 may thus be considered an application of rules in combination with learning from prior data to identify appropriate outputs. Analyzing prior data allows the first machine-learning algorithm 144 and the second machine-learning algorithm 146 to learn patterns of test results and outputs that are generally performed when such test results are observed, for example.

Thus, the first machine-learning algorithm 144 and the second machine-learning algorithm 146 take the form of one or a combination of any of the herein described machine-learning functions, for example.

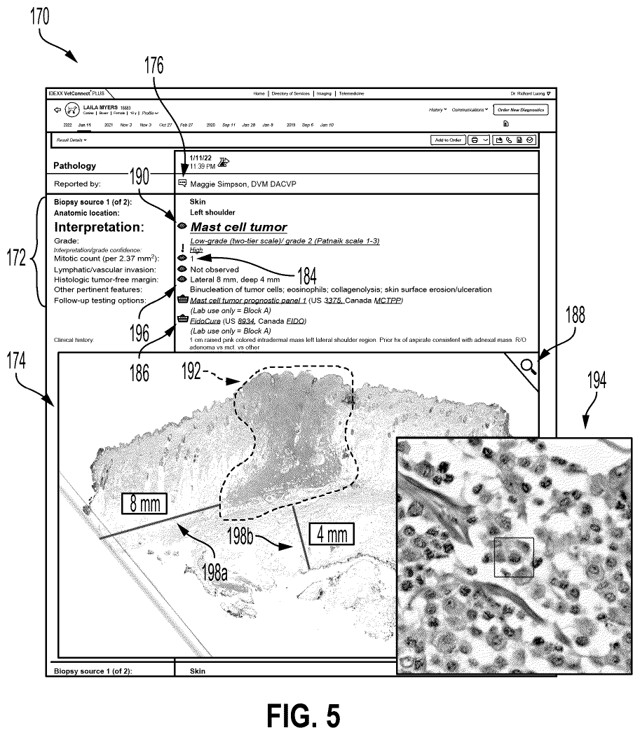

FIG. 5 illustrates another example of the structured output format 170 of the GUI 116 in FIG. 1, according to an example implementation. The diagnosis field 172 in this example includes more details about the diagnosis of the patient based on the data input. In addition, some information in the diagnosis field 172 includes hyperlinks that launch a new and relevant webpage with information and explaining of the terms and diagnosis. In the diagnosis field 172, icons are presented such as icon 184 and a mouse-over the icon 184 generates a pop-up frame with information indicating a confidence indicator that is indicative of a confidence level of the pathology summary based on an amount of differential factors between histologic features of the pathology data and the veterinary pathology training data 150. An example of the confidence indicator includes “High confidence: no reasonable differential exists given histologic features noted by and patient data made available to the pathologist at time of reporting.” Selecting the icon 184 causes a redirect to a website providing information about interpretation/grade confidence.

The diagnosis field 172 also includes shopping cart icons, such as shopping cart icon 186, which when selected will contextually add an associated follow-on test to a patient order. The shopping cart icon 186 is an example of the ordering module 180 selectable to generate an order for follow-on testing, as well as an example of the clinical trials module 182 selectable to schedule a clinical trial.

In FIG. 5, the image field 174 shows images of stained-slides of samples from the patient, and the images can be representative of physically stained-slides or digitally-stained slides. In the image field 174, a magnifying icon 188 is included and selecting the magnifying icon 188 launches an image viewer to display the slide image a full resolution in a new window. Pop-up images and graphic overlays are shown on the stained slide image in the image field 174. To generate the pop-up images and graphic overlays, the GUI 116 receives selection or mouse-over of icons in the diagnosis field 172. For example, upon selection or mouse-over of icon 190, a tumor graphic overlay 192 is shown to annotate the stained image and identify an area of tissue including a tumor. Thus, selection of icons in the diagnosis field creates annotations on the pathology image indicative of histologic tumor free areas and tumor cells, for example.

In another example, upon selection or mouse-over of the icon 184, a pop-up image 194 is generated to illustrate a mitotic figure. In still another example, upon selection or mouse-over of icon 196, dimension graphics 198a-b are illustrated on the stained image to give context of a size of the tumor.

In FIGS. 4 and 5, the contact information module 176 is shown including contact information of a pathologist associated with the pathology data, and selection of mouse-over of the contact information module 176 generates a pop-up frame with information such as “Chat with the reporting pathologist or pathologist-on-duty (POD)” to enable an online chat feature.

The structured output format 170 in FIG. 5 is representative of analysis (or biopsy) of a specific region of the patient, namely, a left shoulder of the patient. When more than one region are analyzed, the structured output format 170 includes a pathology summary of multiple regions or all of the regions.

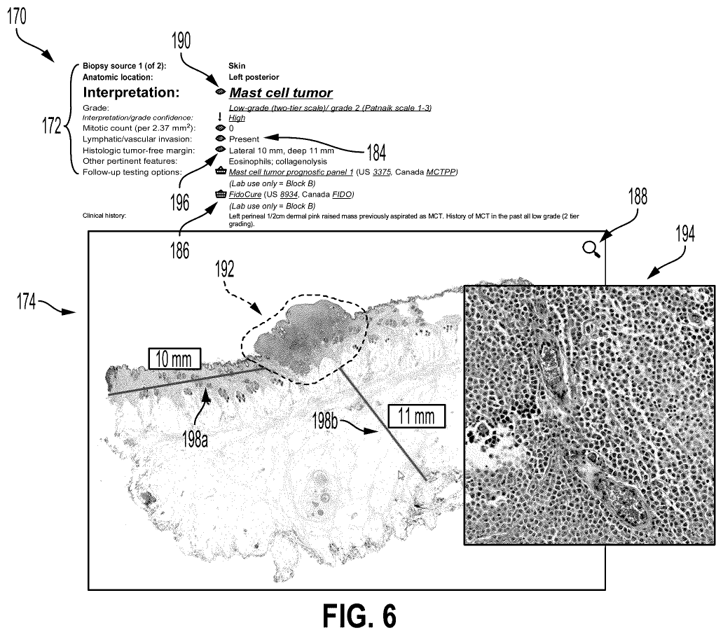

FIG. 6 illustrates another example of the structured output format 170 of the GUI 114 in FIG. 1, according to an example implementation. The information shown in FIG. 6 may be combined into one report with the information in FIG. 5, or the information in FIG. 6 can be provided as a stand-alone report as well.

In FIG. 6, an analysis (or biopsy) of a different region is shown, namely, of a left posterior of the patient. A different analysis of tissue in the image is shown.

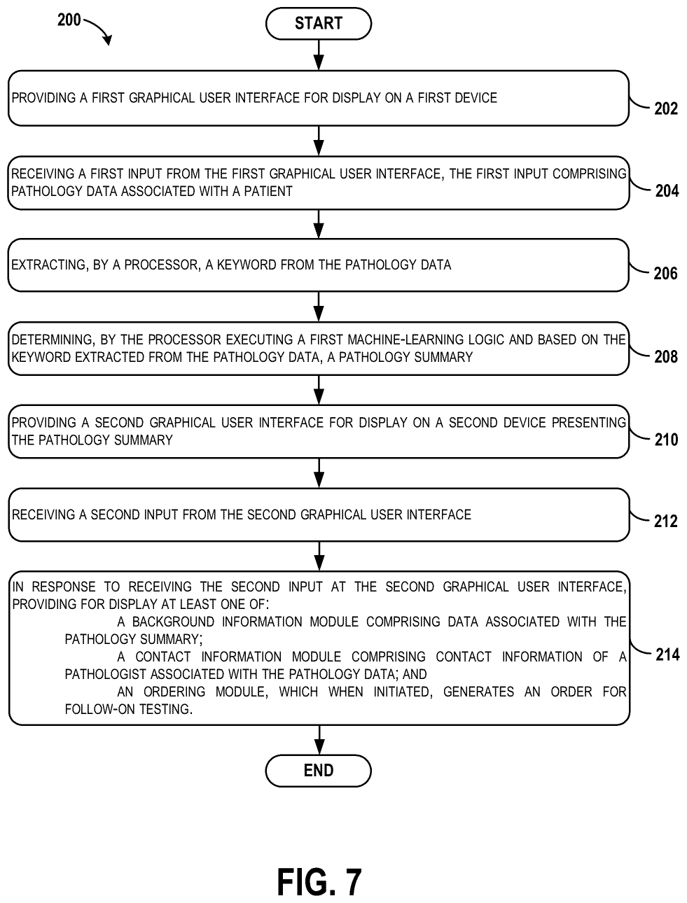

FIG. 7 shows a flowchart of an example of a method 200 for processing pathology data, according to an example implementation. Method 200 shown in FIG. 7 presents an example of a method that could be used with the system 100 shown in FIG. 1, the server 102, shown in FIG. 1, or the computer system 106 shown in FIG. 2, for example. Further, devices or systems may be used or configured to perform logical functions presented in FIG. 7. In some instances, components of the devices and/or systems may be configured to perform the functions such that the components are actually configured and structured (with hardware and/or software) to enable such performance. In other examples, components of the devices and/or systems may be arranged to be adapted to, capable of, or suited for performing the functions, such as when operated in a specific manner. Method 200 may include one or more operations, functions, or actions as illustrated by one or more of blocks 202-214. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

It should be understood that for this and other processes and methods disclosed herein, flowcharts show functionality and operation of one possible implementation of present examples. In this regard, each block or portions of each block may represent a module, a segment, or a portion of program code, which includes one or more instructions executable by a processor for implementing specific logical functions or steps in the process. The program code may be stored on any type of computer readable medium or data storage, for example, such as a storage device including a disk or hard drive. Further, the program code can be encoded on a computer-readable storage media in a machine-readable format, or on other non-transitory media or articles of manufacture. The computer readable medium may include non-transitory computer readable medium or memory, for example, such as computer-readable media that stores data for short periods of time like register memory, processor cache and Random Access Memory (RAM). The computer readable medium may also include non-transitory media, such as secondary or persistent long term storage, like read only memory (ROM), optical or magnetic disks, compact-disc read only memory (CD-ROM), for example. The computer readable media may also be any other volatile or non-volatile storage systems. The computer readable medium may be considered a tangible computer readable storage medium, for example.

In addition, each block or portions of each block in FIG. 7, and within other processes and methods disclosed herein, may represent circuitry that is wired to perform the specific logical functions in the process. Alternative implementations are included within the scope of the examples of the present disclosure in which functions may be executed out of order from that shown or discussed, including substantially concurrent or in reverse order, depending on the functionality involved, as would be understood by those reasonably skilled in the art.

At block 202, the method 200 includes providing a first graphical user interface for display on a first device. In one example, block 202 includes generating a structured template based on the first input provided at the first graphical user interface, and the structured template comprises fields displayed for input in a rules-based manner such that the first graphical user interface displays a field for further input and each subsequent field provided for display is based on the further input provided in a prior field.

At block 204, the method 200 includes receiving a first input from the first graphical user interface, and the first input comprising pathology data associated with a patient. In one example, the pathology data associated with the patient comprises a digitally-stained slide.

At block 206, the method 200 includes extracting, by a processor, a keyword from the pathology data.

At block 208, the method 200 includes determining, by the processor executing a first machine-learning logic and based on the keyword extracted from the pathology data, a pathology summary, and the first machine-learning logic is trained using veterinary pathology training data labeled with corresponding diagnostic results.

In one example, the pathology summary comprises a mitotic count. In some instances, the method 200 also includes receiving a pathology image of the patient, comparing, by the processor, the pathology image of the patient with a reference image, and in response to comparing the pathology image of the patient with the reference image, the processor determining the mitotic count. In one example, the method 200 includes based on the mitotic count, creating, by the processor, a digitally-stained slide based on a prior collected sample of the patient, wherein the digitally-stained slide is a virtual version of a stained slide, and providing within the pathology summary for display on the second graphical user interface an analysis of the digitally-stained slide.

Still further, (as illustrated in FIGS. 5-6) the method 200 optionally includes receiving a pathology image of the patient, creating, by the processor, annotations on the pathology image indicative of histologic tumor free areas and tumor cells, and providing within the pathology summary for display on the second graphical user interface the pathology image of the patient annotated to highlight the tumor cells. In addition, the method 200 optionally includes creating, by the processor, a pop-up image based on a zoomed-in view of the tumor cells, wherein the pop-up image illustrates a mitotic figure, and providing within the pathology summary for display on the second graphical user interface the pop-up image.

In one example, block 208 also includes providing within the pathology summary a confidence indicator that is indicative of a confidence level of the pathology summary based on an amount of differential factors between histologic features of the pathology data and the veterinary pathology training data.

In some examples, determining the pathology summary includes based on an analysis of the first input, the processor determining that a stained slide analysis of a sample from the patient is needed; creating, by the processor, a digitally-stained slide based on a prior collected sample of the patient and the digitally-stained slide is a virtual version of a stained slide; and providing within the pathology summary for display on the second graphical user interface an analysis of the digitally-stained slide. In one example, creating the digitally-stained slide comprises creating the digitally-stained slide based on a mix of stains.

In examples where the digitally-stained slide is created, the method 200 optionally includes determining, by the processor executing a second machine-learning logic, the analysis of the digitally-stained slide, and the second machine-learning logic is trained using images of a plurality of physically stained slides labeled with attributes for a stain used on a sample.

At block 210, the method 200 includes providing a second graphical user interface for display on a second device presenting the pathology summary. In one example, at block 210, the processor selects the second device to provide the second graphical user interface from among a pool of devices. The second device is determined, by the processor, to be associated with a patient undergoing treatment and that is the patient for which the pathology data has been input at block 204. Thus, in one example, the pathology data input includes a patient identifier, and the processor uses the patient identifier as a basis to select the second device. The second device is pre-registered as being associated with the patient identifier so that the pathology summary is digitally routed, via the network 104, to the correct second device.

At block 212, the method 200 includes receiving a second input from the second graphical user interface.

At block 214, the method 200 includes in response to receiving the second input at the second graphical user interface, providing for display at least one of: a background information module comprising data associated with the pathology summary; a contact information module comprising contact information of a pathologist associated with the pathology data; and an ordering module, which when initiated, generates an order for follow-on testing. In some examples, upon receiving the second input at the second graphical user interface, the method 200 includes updating a display of the second graphical user interface to include one of new information, a new window frame, a pop-up window, animation effects, or other graphical and interactive features.

In one example, the method 200 optionally also includes creating, by the processor, a digitally-stained slide based on a prior collected sample of the patient, wherein the digitally-stained slide is a virtual version of a stained slide, and providing within the pathology summary for display on the second graphical user interface an analysis of the digitally-stained slide. The digitally-stained slide is created, by the processor executing a second machine-learning logic, and the second machine-learning logic is trained using images of a plurality of physically stained slides labeled with attributes for a stain used on a sample.

With reference to FIG. 2, and throughout the disclosure, some components are described as “modules,” “engines”, “models”, or “generators” and such components include or take a form of a general purpose or special purpose hardware (e.g., general or special purpose processors), firmware, and/or software embodied in a non-transitory computer-readable (storage) medium for execution by one or more processors to perform described functionality.

The description of the different advantageous arrangements has been presented for purposes of illustration and description, and is not intended to be exhaustive or limited to the examples in the form disclosed. Many modifications and variations will be apparent to those of ordinary skill in the art. Further, different advantageous examples may describe different advantages as compared to other advantageous examples. The example or examples selected are chosen and described in order to explain the principles of the examples, the practical application, and to enable others of ordinary skill in the art to understand the disclosure for various examples with various modifications as are suited to the particular use contemplated.

Different examples of the system(s), device(s), and method(s) disclosed herein include a variety of components, features, and functionalities. It should be understood that the various examples of the system(s), device(s), and method(s) disclosed herein may include any of the components, features, and functionalities of any of the other examples of the system(s), device(s), and method(s) disclosed herein in any combination or any sub-combination, and all of such possibilities are intended to be within the scope of the disclosure.

Thus, examples of the present disclosure relate to enumerated clauses (ECs) listed below in any combination or any sub-combination.

EC 1 is a computer-implemented method for processing pathology data, the method comprising: providing a first graphical user interface for display on a first device; receiving a first input from the first graphical user interface, the first input comprising pathology data associated with a patient; extracting, by a processor, a keyword from the pathology data; determining, by the processor executing a first machine-learning logic and based on the keyword extracted from the pathology data, a pathology summary, wherein the first machine-learning logic is trained using veterinary pathology training data labeled with corresponding diagnostic results; providing a second graphical user interface for display on a second device presenting the pathology summary; and receiving a second input from the second graphical user interface; and in response to receiving the second input at the second graphical user interface, providing for display at least one of: a background information module comprising data associated with the pathology summary; a contact information module comprising contact information of a pathologist associated with the pathology data; and an ordering module, which when initiated, generates an order for follow-on testing.

EC 2 is the method of EC 1, wherein: the pathology data associated with the patient comprises a digitally-stained slide.

EC 3 is the method of any of ECs 1-2, wherein: wherein the pathology summary comprises a mitotic count.

EC 4 is the method of any of ECs 1-3, further comprising: receiving a pathology image of the patient; comparing, by the processor, the pathology image of the patient with a reference image; and in response to comparing the pathology image of the patient with the reference image, the processor determining the mitotic count.

EC 5 is the method of any of ECs 1-4, further comprising: based on the mitotic count, creating, by the processor, a digitally-stained slide based on a prior collected sample of the patient, wherein the digitally-stained slide is a virtual version of a stained slide; and providing within the pathology summary for display on the second graphical user interface an analysis of the digitally-stained slide.

EC 6 is the method of any of ECs 1-5, further comprising: receiving a pathology image of the patient; creating, by the processor, annotations on the pathology image indicative of histologic tumor free areas and tumor cells; and providing within the pathology summary for display on the second graphical user interface the pathology image of the patient annotated to highlight the tumor cells.

EC 7 is the method of any of ECs 1-6, further comprising: creating, by the processor, a pop-up image based on a zoomed-in view of the tumor cells, wherein the pop-up image illustrates a mitotic figure; and providing within the pathology summary for display on the second graphical user interface the pop-up image.

EC 8 is the method of any of ECs 1-7, further comprising: providing within the pathology summary a confidence indicator that is indicative of a confidence level of the pathology summary based on an amount of differential factors between histologic features of the pathology data and the veterinary pathology training data.

EC 9 is the method of any of ECs 1-8, further comprising: generating a structured template based on the first input provided at the first graphical user interface, wherein the structured template comprises fields displayed for input in a rules-based manner such that the first graphical user interface displays a field for further input and each subsequent field provided for display is based on the further input provided in a prior field.

EC 10 is the method of any of ECs 1-9, further comprising: creating, by the processor, a digitally-stained slide based on a prior collected sample of the patient, wherein the digitally-stained slide is a virtual version of a stained slide; and providing within the pathology summary for display on the second graphical user interface an analysis of the digitally-stained slide.

EC 11 is the method of any of ECs 1-10, further comprising: creating, by the processor executing a second machine-learning logic, the digitally-stained slide, wherein the second machine-learning logic is trained using images of a plurality of physically stained slides labeled with attributes for a stain used on a sample.

EC 12 is the method of any of ECs 1-11, further comprising: based on an analysis of the first input, the processor determining that a stained slide analysis of a sample from the patient is needed; creating, by the processor, a digitally-stained slide based on a prior collected sample of the patient, wherein the digitally-stained slide is a virtual version of a stained slide; and providing within the pathology summary for display on the second graphical user interface an analysis of the digitally-stained slide.

EC 13 is the method of any of ECs 1-12, wherein: creating the digitally-stained slide comprises: creating the digitally-stained slide based on a mix of stains.

EC 14 is the method of any of ECs 1-13, further comprising: determining, by the processor executing a second machine-learning logic, the analysis of the digitally-stained slide, wherein the second machine-learning logic is trained using images of a plurality of physically stained slides labeled with attributes for a stain used on a sample.

EC 15 is a server comprising: one or more processors; and non-transitory computer readable medium having stored therein instructions that when executed by the one or more processors, causes the server to perform functions comprising: providing a first graphical user interface for display on a first device; receiving a first input from the first graphical user interface, the first input comprising pathology data associated with a patient; extracting a keyword from the pathology data; determining, by executing a first machine-learning logic and based on the keyword extracted from the pathology data, a pathology summary, wherein the first machine-learning logic is trained using veterinary pathology training data labeled with corresponding diagnostic results; providing a second graphical user interface for display on a second device presenting the pathology summary; and receiving a second input from the second graphical user interface; and in response to receiving the second input at the second graphical user interface, providing for display at least one of: a background information module comprising data associated with the pathology summary; a contact information module comprising contact information of a pathologist associated with the pathology data; and an ordering module, which when initiated, generates an order for follow-on testing.

EC 16 is the server of EC 15, wherein the pathology summary comprises a mitotic count and the functions further comprise: receiving a pathology image of the patient; comparing the pathology image of the patient with a reference image; and in response to comparing the pathology image of the patient with the reference image, determining the mitotic count.

EC 17 is the server of any of ECs 15-16, wherein the functions further comprise: based on the mitotic count, creating a digitally-stained slide based on a prior collected sample of the patient, wherein the digitally-stained slide is a virtual version of a stained slide; and providing within the pathology summary for display on the second graphical user interface an analysis of the digitally-stained slide.

EC 18 is a non-transitory computer readable medium having stored thereon instructions, that when executed by one or more processors of a computing device, cause the computing device to perform functions comprising: providing a first graphical user interface for display on a first device; receiving a first input from the first graphical user interface, the first input comprising pathology data associated with a patient; extracting a keyword from the pathology data; determining, by executing a first machine-learning logic and based on the keyword extracted from the pathology data, a pathology summary, wherein the first machine-learning logic is trained using veterinary pathology training data labeled with corresponding diagnostic results; providing a second graphical user interface for display on a second device presenting the pathology summary; and receiving a second input from the second graphical user interface; and in response to receiving the second input at the second graphical user interface, providing for display at least one of: a background information module comprising data associated with the pathology summary; a contact information module comprising contact information of a pathologist associated with the pathology data; and an ordering module, which when initiated, generates an order for follow-on testing.

EC 19 is the non-transitory computer readable medium of EC 18, wherein: the functions further comprise: creating a digitally-stained slide based on a prior collected sample of the patient, wherein the digitally-stained slide is a virtual version of a stained slide; and providing within the pathology summary for display on the second graphical user interface an analysis of the digitally-stained slide.

EC 20 is the non-transitory computer readable medium of any of ECs 18-19, wherein: wherein the functions further comprise: creating, by executing a second machine-learning logic, the digitally-stained slide, wherein the second machine-learning logic is trained using images of a plurality of physically stained slides labeled with attributes for a stain used on a sample.

By the term “substantially” and “about” used herein, it is meant that the recited characteristic, parameter, or value need not be achieved exactly, but that deviations or variations, including for example, tolerances, measurement error, measurement accuracy limitations and other factors known to skill in the art, may occur in amounts that do not preclude the effect the characteristic was intended to provide. The terms “substantially” and “about” represent the inherent degree of uncertainty that may be attributed to any quantitative comparison, value, measurement, or other representation. The terms “substantially” and “about” are also utilized herein to represent the degree by which a quantitative representation may vary from a stated reference without resulting in a change in the basic function of the subject matter at issue.

It is noted that one or more of the following claims utilize the term “wherein” as a transitional phrase. For the purposes of defining the present invention, it is noted that this term is introduced in the claims as an open-ended transitional phrase that is used to introduce a recitation of a series of characteristics of the structure and should be interpreted in like manner as the more commonly used open-ended preamble term “comprising.”

What is claimed is:

1. A computer-implemented method for processing pathology data, the method comprising:
providing a first graphical user interface for display on a first device;
receiving a first input from the first graphical user interface, the first input comprising pathology data associated with a patient;
extracting, by a processor, a keyword from the pathology data;
determining, by the processor executing a first machine-learning logic and based on the keyword extracted from the pathology data, a pathology summary, wherein the first machine-learning logic is trained using veterinary pathology training data labeled with corresponding diagnostic results;
providing a second graphical user interface for display on a second device presenting the pathology summary; and
receiving a second input from the second graphical user interface; and
in response to receiving the second input at the second graphical user interface, providing for display at least one of:
a background information module comprising data associated with the pathology summary;
a contact information module comprising contact information of a pathologist associated with the pathology data; and
an ordering module, which when initiated, generates an order for follow-on testing.

2. The computer-implemented method of claim 1, wherein the pathology data associated with the patient comprises a digitally-stained slide.

3. The computer-implemented method of claim 1, wherein the pathology summary comprises a mitotic count.

4. The computer-implemented method of claim 3, further comprising:
receiving a pathology image of the patient;
comparing, by the processor, the pathology image of the patient with a reference image; and
in response to comparing the pathology image of the patient with the reference image, the processor determining the mitotic count.

5. The computer-implemented method of claim 3, further comprising:
based on the mitotic count, creating, by the processor, a digitally-stained slide based on a prior collected sample of the patient, wherein the digitally-stained slide is a virtual version of a stained slide; and
providing within the pathology summary for display on the second graphical user interface an analysis of the digitally-stained slide.

6. The computer-implemented method of claim 3, further comprising:
receiving a pathology image of the patient;
creating, by the processor, annotations on the pathology image indicative of histologic tumor free areas and tumor cells; and
providing within the pathology summary for display on the second graphical user interface the pathology image of the patient annotated to highlight the tumor cells.

7. The computer-implemented method of claim 6, further comprising:
creating, by the processor, a pop-up image based on a zoomed-in view of the tumor cells, wherein the pop-up image illustrates a mitotic figure; and
providing within the pathology summary for display on the second graphical user interface the pop-up image.

8. The computer-implemented method of claim 1, further comprising:
providing within the pathology summary a confidence indicator that is indicative of a confidence level of the pathology summary based on an amount of differential factors between histologic features of the pathology data and the veterinary pathology training data.

9. The computer-implemented method of claim 1, further comprising:
generating a structured template based on the first input provided at the first graphical user interface, wherein the structured template comprises fields displayed for input in a rules-based manner such that the first graphical user interface displays a field for further input and each subsequent field provided for display is based on the further input provided in a prior field.

10. The computer-implemented method of claim 1, further comprising:
creating, by the processor, a digitally-stained slide based on a prior collected sample of the patient, wherein the digitally-stained slide is a virtual version of a stained slide; and
providing within the pathology summary for display on the second graphical user interface an analysis of the digitally-stained slide.

11. The computer-implemented method of claim 10, further comprising:
creating, by the processor executing a second machine-learning logic, the digitally-stained slide, wherein the second machine-learning logic is trained using images of a plurality of physically stained slides labeled with attributes for a stain used on a sample.

12. The computer-implemented method of claim 1, further comprising:
based on an analysis of the first input, the processor determining that a stained slide analysis of a sample from the patient is needed;
creating, by the processor, a digitally-stained slide based on a prior collected sample of the patient, wherein the digitally-stained slide is a virtual version of a stained slide; and
providing within the pathology summary for display on the second graphical user interface an analysis of the digitally-stained slide.

13. The computer-implemented method of claim 12, wherein creating the digitally-stained slide comprises:
creating the digitally-stained slide based on a mix of stains.

14. The computer-implemented method of claim 12, further comprising:
determining, by the processor executing a second machine-learning logic, the analysis of the digitally-stained slide, wherein the second machine-learning logic is trained using images of a plurality of physically stained slides labeled with attributes for a stain used on a sample.

15. A server comprising:
one or more processors; and
non-transitory computer readable medium having stored therein instructions that when executed by the one or more processors, causes the server to perform functions comprising:
providing a first graphical user interface for display on a first device;
receiving a first input from the first graphical user interface, the first input comprising pathology data associated with a patient;
extracting a keyword from the pathology data;
determining, by executing a first machine-learning logic and based on the keyword extracted from the pathology data, a pathology summary, wherein the first machine-learning logic is trained using veterinary pathology training data labeled with corresponding diagnostic results;
providing a second graphical user interface for display on a second device presenting the pathology summary; and
receiving a second input from the second graphical user interface; and
in response to receiving the second input at the second graphical user interface, providing for display at least one of:
a background information module comprising data associated with the pathology summary;
a contact information module comprising contact information of a pathologist associated with the pathology data; and
an ordering module, which when initiated, generates an order for follow-on testing.

16. The server of claim 15, wherein the pathology summary comprises a mitotic count and the functions further comprise:
receiving a pathology image of the patient;
comparing the pathology image of the patient with a reference image; and
in response to comparing the pathology image of the patient with the reference image, determining the mitotic count.

17. The server of claim 16, wherein the functions further comprise:
based on the mitotic count, creating a digitally-stained slide based on a prior collected sample of the patient, wherein the digitally-stained slide is a virtual version of a stained slide; and
providing within the pathology summary for display on the second graphical user interface an analysis of the digitally-stained slide.

18. A non-transitory computer readable medium having stored thereon instructions, that when executed by one or more processors of a computing device, cause the computing device to perform functions comprising:
providing a first graphical user interface for display on a first device;
receiving a first input from the first graphical user interface, the first input comprising pathology data associated with a patient;
extracting a keyword from the pathology data;
determining, by executing a first machine-learning logic and based on the keyword extracted from the pathology data, a pathology summary, wherein the first machine-learning logic is trained using veterinary pathology training data labeled with corresponding diagnostic results;
providing a second graphical user interface for display on a second device presenting the pathology summary; and
receiving a second input from the second graphical user interface; and
in response to receiving the second input at the second graphical user interface, providing for display at least one of:
a background information module comprising data associated with the pathology summary;
a contact information module comprising contact information of a pathologist associated with the pathology data; and
an ordering module, which when initiated, generates an order for follow-on testing.

19. The non-transitory computer readable medium of claim 18, wherein the functions further comprise:
creating a digitally-stained slide based on a prior collected sample of the patient, wherein the digitally-stained slide is a virtual version of a stained slide; and
providing within the pathology summary for display on the second graphical user interface an analysis of the digitally-stained slide.

20. The non-transitory computer readable medium of claim 19, wherein the functions further comprise:
creating, by executing a second machine-learning logic, the digitally-stained slide, wherein the second machine-learning logic is trained using images of a plurality of physically stained slides labeled with attributes for a stain used on a sample.
